(19) United States
(12) Patent Application Publication
(10) Pub. No.: US 2016/0174852 A1
(43) Pub. Date: Jun. 23, 2016

(54) WEIGHT-BEARING BIOFEEDBACK DEVICES

(71) Applicant: Quanttus, Inc., Cambridge, MA (US)

(72) Inventors: David Da He, Cambridge, MA (US); Shahid Azim, Cambridge, MA (US)

(73) Assignee: Quanttus, Inc., Cambridge, MA (US)

(21) Appl. No.: 14/943,475

(22) Filed: Nov. 17, 2015

Related U.S. Application Data
(60) Provisional application No. 62/094,647, filed on Dec. 19, 2014.

(51) Int. Cl. A61B 5/0205; A61B 5/029; G01G 19/50; A61B 5/00

(52) U.S. Cl. CPC A61B 5/02055; A61B 5/6807; A61B 5/6892; A61B 5/6889; A61B 5/6891; A61B 5/029; A61B 5/7278; A61B 5/4872; G01G 19/50; A61B 2562/0219; A61B 2562/0247; A61B 2562/0261; A61B 2562/0214; A61B 2560/0468; A61B 2562/0233

(57) ABSTRACT

A weight-bearing device comprising: a weight-bearing surface configured to bear the weight of a subject; a first sensor module disposed in the device, the first sensor module configured to measure information about pulse waves propagating through blood in the subject, the subject located in contact with the weight-bearing surface; a second sensor module disposed in the device, the second sensor module configured to measure information about a motion of the subject; and a processing device configured to: receive a first dataset representing time-varying information about at least one pulse wave propagating through blood in the subject, wherein the time-varying information about the at least one pulse wave is measured using the first sensor module; receive a second dataset representing information about a time-varying motion of the subject, wherein the information about the time-varying motion is measured using the second sensor module; identify a first point in the first dataset, the first point representing an arrival time of the pulse wave at a first body part of the subject; identify a second point in the second dataset, the second point representing an earlier time at which the pulse wave traverses a second body part of the subject; and compute a pulse transit time (PTT) as a difference between the first and second points, the PTT representing a time taken by the pulse wave to travel from the second body part to the first body part of the subject.

### CLAIM OF PRIORITY

This application claims priority to U.S. Provisional Patent Application Ser. No. 62/094,647, filed on Dec. 19, 2014, the entire contents of which are hereby incorporated by reference.

### TECHNICAL FIELD

This document relates to weight-bearing biofeedback devices.

### BACKGROUND

Various types of sensors can be used for sensing biometric parameters.

### SUMMARY

In one aspect, a weight-bearing device includes a weight-bearing surface configured to bear the weight of a subject. The weight-bearing device also includes a first sensor module disposed in the device. The first sensor module is configured to measure information about pulse waves propagating through blood in the subject. The subject is located in contact with the weight-bearing surface. The weight-bearing device also includes a second sensor module disposed in the device. The second sensor module is configured to measure information about a motion of the subject. The weight-bearing device also includes a processing device. The processing device is configured to receive a first dataset representing time-varying information about at least one pulse wave propagating through blood in the subject. The time-varying information about the at least one pulse wave is measured using the first sensor module. The processing device is also configured to receive a second dataset representing information about a time-varying motion of the subject. The information about the time-varying motion is measured using the second sensor module. The processing device is also configured to identify a first point in the first dataset. The first point represents an arrival time of the pulse wave at a first body part of the subject. The processing device is also configured to identify a second point in the second dataset. The second point represents an earlier time at which the pulse wave traverses a second body part of the subject. The processing device is also configured to compute a pulse transit time (PTT) as a difference between the first and second points. The PTT represents a time taken by the pulse wave to travel from the second body part to the first body part of the subject.

Implementations can include one or more of the following features.

In some implementations, the weight-bearing surface is flexible.

In some implementations, the second sensor module includes a strain gauge.

In some implementations, the second sensor module includes a motion sensor.

In some implementations, the motion sensor includes one or both of an accelerometer and a gyroscope.

In some implementations, the weight-bearing surface is rigid.

In some implementations, the second sensor module includes a pressure sensor.

In some implementations, the weight-bearing device also includes a mechanism affixed to an underside of the weight-bearing surface. The mechanism is configured to permit the weight-bearing surface to depress.

In some implementations, the mechanism is a spring.

In some implementations, the second sensor module includes a motion sensor.

In some implementations, the motion sensor includes one or both of an accelerometer and a gyroscope.

In some implementations, the first sensor module includes a light source and an optical sensor.

In some implementations, the light source is an LED.

In some implementations, the optical sensor is a photodiode.

In some implementations, the first sensor module includes an impedance sensor.

In some implementations, the impedance sensor includes two electrodes positioned less than 4 inches of each other.

In some implementations, the electrodes are positioned such that a part of the skin of the subject makes direct contact with both of the electrodes when the weight-bearing surface bears the weight of the subject.

In some implementations, the electrodes are positioned such that a foot of the subject makes direct contact with both of the electrodes when the weight-bearing surface bears the weight of the subject.

In some implementations, the impedance sensor includes two electrodes positioned greater than or equal to 4 inches from each other.

In some implementations, the electrodes are positioned such that a first foot of the subject makes contact with one of the electrodes and a second foot of the subject makes contact with the other electrode when the weight-bearing surface bears the weight of the subject.

In some implementations, the information about pulse waves propagating through blood in the subject comprises photoplethysmographic (PPG) data.

In some implementations, the information about pulse waves propagating through blood in the subject comprises bio-impedance data.

In some implementations, the information about a motion of the subject comprises ballistocardiogram (BCG) data.

In some implementations, the information about a motion of the subject comprises seismocardiogram (SCG) data.

In some implementations, identifying the first point in the first dataset includes identifying a reference point within the first dataset.

In some implementations, the reference point is a local maximum, a local minimum, a zero-crossing, or a local maximum of a first derivative within the first dataset.

In some implementations, the reference point is within an expected range of one or both of time and amplitude.

In some implementations, identifying the second point in the second dataset includes identifying a reference point within the second dataset.

In some implementations, the reference point is a local maximum, a local minimum, a zero-crossing, or a local maximum of a first derivative within the first dataset.

In some implementations, the reference point is within an expected range of one or both of time and amplitude.

In some implementations, the weight-bearing surface is substantially flat.

In some implementations, at least one of the first sensor module and the second sensor module is attached to the weight-bearing surface.

In some implementations, the weight-bearing surface is configured to directly contact the subject when the weight-bearing surface bears the weight of the subject.

In some implementations, the device is a weight scale.

In some implementations, the device is integrated into a floor.

In some implementations, the device is a floor tile.

In some implementations, the device is a bed.

In some implementations, the device is a yoga mat.

In some implementations, the device is a shoe.

In some implementations, the weight-bearing surface is a sole of the shoe.

In some implementations, at least one of the first sensor module and the second sensor module is attached to the sole of the shoe.

In some implementations, the device is a chair.

In some implementations, the second sensor module includes a sensor for measuring a weight of the subject.

In some implementations, the processing device is further configured to determine one or more of a blood pressure, a heart rate, a respiratory rate, a blood oxygen level, a stroke volume, a cardiac output, and a temperature of the subject.

In some implementations, the processing device determines the heart rate, the respiratory rate, the stroke volume, and the cardiac output based on the information measured by the second sensor module without using the information measured by the first sensor module.

In some implementations, the second sensor module is configured to measure a weight of the subject.

In some implementations, the weight-bearing device is configured to measure a body composition of the subject.

In some implementations, the body composition of the subject includes a fat content of the subject.

In another aspect, a device includes a weight-bearing surface configured to bear the weight of a subject. The device also includes a first sensor module and a second sensor module each disposed in the device. The first sensor module and the second sensor module are each configured to measure information about pulse waves propagating through blood in the subject. The subject is located in contact with the weight-bearing surface. The device also includes a processing device. The processing device is configured to receive a first dataset representing time-varying information about at least one pulse wave propagating through blood in the subject. The time-varying information about the at least one pulse wave is measured using the first sensor module. The processing device is also configured to receive a second dataset representing time-varying information about the at least one pulse wave propagating through blood in the subject. The time-varying information about the at least one pulse wave is measured using the second sensor module. The processing device is also configured to identify a first point in the first dataset. The first point represents an arrival time of the pulse wave at a first body part of the subject. The processing device is also configured to identify a second point in the second dataset. The second point represents an arrival time of the pulse wave at a second body part of the subject. The processing device is also configured to compute a pulse transit time (PTT) as a difference between the first and second points. The PTT represents a time taken by the pulse wave to travel from the first body part to the second body part of the subject.

Implementations can include one or more of the following features.

In some implementations, at least one of the first sensor module and the second sensor module includes a light source and an optical sensor.

In some implementations, the light source is an LED.

In some implementations, the optical sensor is a photodiode.

In some implementations, at least one of the first sensor module and the second sensor module includes an impedance sensor.

In some implementations, the impedance sensor includes two electrodes positioned less than 4 inches of each other.

In another aspect, a device includes a weight-bearing surface configured to bear the weight of a subject. The device also includes a first sensor module disposed in the device. The first sensor module is configured to measure information about pulse waves propagating through blood in the subject. The subject is located in contact with the weight-bearing surface. The device also includes a second sensor module disposed in the device. The second sensor module is configured to measure information about electrical signals related to the heart of the subject. The device also includes a processing device. The processing device is configured to receive a first dataset representing time-varying information about at least one pulse wave propagating through blood in the subject. The time-varying information about the at least one pulse wave is measured using the first sensor module. The processing device is also configured to receive a second dataset representing time-varying information about electrical signals related to the heart of the subject. The time-varying information about electrical signals related to the heart of the subject is measured using the second sensor module. The processing device is also configured to identify a first point in the first dataset. The first point represents an arrival time of the pulse wave at a body part of the subject. The processing device is also configured to identify a second point in the second dataset. The second point represents an earlier time at which the heart of the subject is depolarized. The pulse wave is originated from the heart of the subject in response to the depolarization. The processing device is also configured to compute a pulse arrival time (PAT) as a difference between the first and second points. The PAT represents an elapsed time between the pulse wave being originated and the pulse wave arriving at the body part of the subject.

In some implementations, the PAT represents an approximate time taken by the pulse wave to travel from the heart of the subject to the body part of the subject.

Implementations can include one or more of the following advantages.

Blood pressure and/or other biometric parameters may be measured based on collected data without the need for constraining accessories such as cuffs or leads. Vital signs can be measured, using a comfortable and unobtrusive weight-bearing device such as a mat, weight-scale, chair, bed, or yoga mat. As such, the technology described herein can be used for regular (e.g., continuous) measurements of biometric parameters under substantially similar conditions. For example, if the sensors for measuring the biometric parameters are disposed on a bathroom mat, a user may be able to obtain measurements regularly, and under substantially similar conditions (e.g., after waking up every morning). This can facilitate easy measurement, and meaningful tracking of the biometric parameters.

Other aspects, features, and advantages of the invention will be apparent from the description and drawings, and from the claims.

### DESCRIPTION OF DRAWINGS

FIG. 1 shows an example of a device that includes a weight-bearing surface, a strain gauge, and a sensor insert.

FIG. 2 is a perspective view of the device of FIG. 1 showing a deformation of the weight-bearing surface in response to an applied weight.

FIG. 3 is an example of a sensor insert included in the device FIG. 1.

FIG. 4 shows examples of pulse transit times (PTT) derived using BCG and PPG data.

FIG. 5 is a flowchart depicting an example process of determining PTT based on BCG and PPG data.

FIG. 6 shows an example of a weight-bearing device that includes a motion sensor.

FIG. 7 shows an example of a weight-bearing device that includes springs disposed beneath a rigid weight-bearing surface.

FIGS. 8 and 9 show examples of weight-bearing devices that include an impedance sensor.

FIG. 10 shows an example of a bed that includes a weight-bearing surface, a strain gauge, and a sensor insert.

FIG. 11 shows an example of a shoe that includes a weight-bearing surface, a strain gauge, and a sensor insert.

FIG. 12 shows an example of a chair that includes a weight-bearing surface, a strain gauge, and a sensor insert.

FIG. 13 shows an example of a yoga mat that includes a weight-bearing surface, a strain gauge, and a sensor insert.

FIG. 14 is an example of a block diagram of a computer system.

### DETAILED DESCRIPTION

This document describes devices that can collect various types of data used in measuring and/or deriving one or more health related parameters. Examples of such data include motioncardiogram (MoCG) data (which is related to ballistocardiogram (BCG) data), photoplethysmographic (PPG) data, and bio-impedance data. In some cases, the devices can be configured to measure various biometric parameters (e.g., blood pressure, heart rate, respiratory rate, blood oxygen level, stroke volume, cardiac output, and temperature) based on the collected data (e.g., MoCG data, the PPG data, and the bio-impedance data).

PPG data can be optically obtained via a plethysmogram, a volumetric measurement of the vasculature. PPG can be obtained, for example, using an optical device which illuminates the skin and measures changes in light absorption. With each cardiac cycle the heart pumps blood resulting in a pulse wave within the vasculature. This can cause time-varying changes in the volume of the vasculature. The changes can be detected, for example, by illuminating the skin with light from a light-emitting diode (LED) and then measuring the amount of light either transmitted or reflected to a detector such as a photodiode. Each cardiac cycle can therefore be represented as a pattern of crests and troughs of a PPG waveform, with each crest related to the arrival of a pulse wave at a particular position of a subject's body. The shape of the PPG waveform may differ from subject to subject, and may vary with the location and manner in which the waveform is recorded. For example, a PPG waveform recorded from a foot of the subject may have a different shape than a PPG waveform recorded from a finger of the subject.

Bio-impedance data can also be used to determine the arrival of a pulse wave at a particular position of the subject's body. Bio-impedance data can be obtained, for example, by an impedance sensor such as a galvanic skin resistance sensor that includes electrodes. A voltage or a current is applied across a particular portion of the subject's body and the resultant current or voltage is measured. The current seeks the path of least resistance, which is through the blood of the subject. The voltage and current values can be used to determine the impedance of the blood. With each cardiac cycle, the heart pumps blood resulting in a pressure pulse wave within the vasculature. This causes time-varying changes in the volume of the vasculature. These changes in blood volume result in corresponding changes in the measured blood impedance. Each cardiac cycle is therefore represented as a pattern of crests and troughs of a bio-impedance waveform, with each crest related to the arrival of a pulse wave at a particular position of the subject's body. The shape of the bio-impedance waveform differs from subject to subject, and varies with the location and manner in which the waveform is recorded.

The MoCG is an example of a motion of the subject. For example, MoCG is a pulsatile motion signal of the body measurable, for example, by a strain sensor or a motion sensor such as an accelerometer or a gyroscope. The pulsatile motion signal results from a mechanical motion of portions of the body. The mechanical motion of portions of the body occurs in response to the mechanical motion of the heart. The pulsatile motion is a mechanical reaction of the body to the internal flow of blood and is externally measurable. The MoCG signal therefore corresponds to, but is delayed from, the heartbeat. Various points of the MoCG signal are related to times at which pulse waves traverse a particular position of the subject's body (typically near the subject's heart, e.g., an artery extending from the heart such as the aorta). MoCG data can be used to calculate various biometric parameters such as stroke volume. In some implementations, the amplitude of the MoCG signal corresponds to stroke volume.

Some biometric measurements can be determined by measuring the speed that a pulse wave travels through the subject's body. For example, in the context of determining the blood pressure of the subject, a change in blood pressure will directly result in a change in the speed that the pulse wave travels. In order to measure this speed, two points in time are needed. The first point in time is the time when the pulse wave arrives at a first position of the subject's body, and the second point in time is the time when the pulse wave arrives at a second position of the subject's body. The actual time it takes for a pulse wave to travel from the first position of the subject's body to the second position of the subject's body is called the Pulse Transit Time (PTT). As such, the difference between the first point in time and the second point in time is the PTT.

The PTT can represent the time it takes for a pulse wave to travel from a position near the subject's heart to a position away from the subject's heart. The first point in time can represent an arrival time of the pulse wave at a position of the subject's body that is located some distance from the subject's heart. The first point in time can be determined based on PPG and/or bio-impedance data. An optical device (for collecting PPG data) and/or an impedance sensor (for collecting bio-impedance data) can be located near the position of the subject's body where the pulse wave arrives.

The second point in time can represent an earlier time at which the pulse wave traversed a position of the subject's body that is located near the subject's heart. The second point in time can be determined based on MoCG data. A motion sensor for collecting pulsatile motion data can be located remote from the position of the subject's body where the pulse wave traverses. The motion sensor can be located near the optical device and/or the impedance sensor.

Described herein are weight-bearing biofeedback devices that can collect various types of data from a subject's body (e.g., MoCG, PPG, and bio-impedance data) and perform biometric measurements based on the collected data. The biometric measurements can be used for monitoring health related parameters, as well as for diagnosing conditions and predicting an onset of such conditions. In some cases, the device can include a weight-bearing surface configured to bear the weight of a subject, a first sensor module configured to measure information about pulse waves propagating through blood in the subject, and a second sensor module configured to measure information about a motion of the subject. The device can include a processing device configured to compute, based on the measured information, a PTT that represents the time it takes for a pulse wave to travel from one body part of the subject to another body part of the subject. In some implementations, the device can be configured to provide the measured information to a remote computing device such as a mobile device or server for the remote computing device to derive health information about the subject based on the measured information.

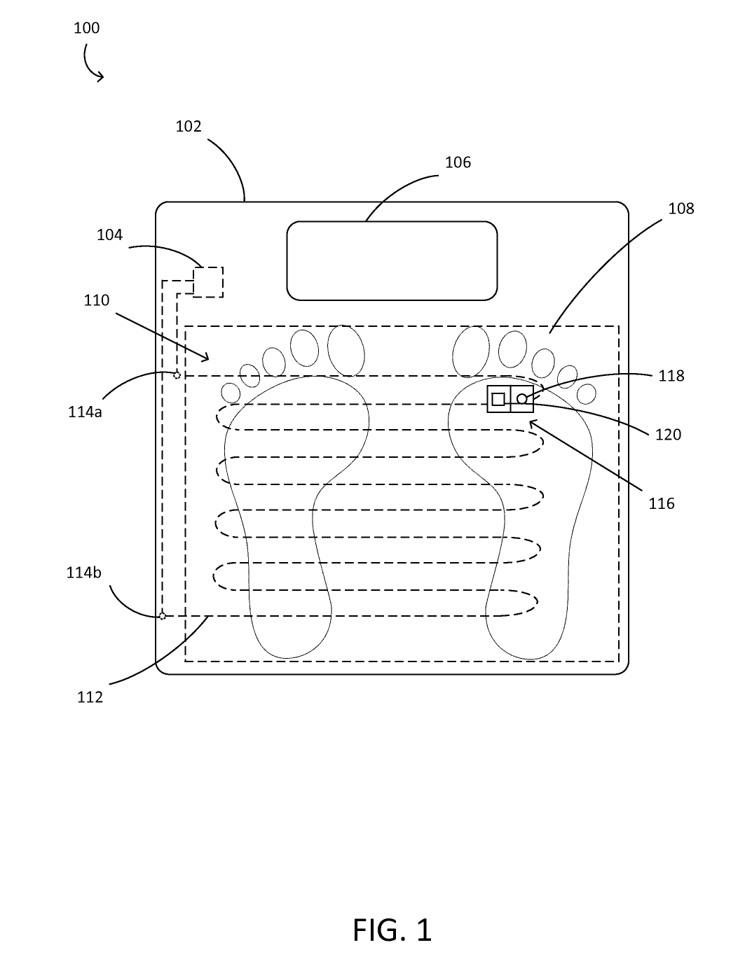

FIG. 1 shows a weight scale 100 as an example of a weight-bearing device that can collect BCG and PPG data and perform various biometric measurements based on the collected data. The weight scale 100 includes a housing 102 for holding internal components of the weight scale 100, such as a processor 104 that is disposed in the housing 102. In some implementations, the weight scale 100 also includes a display 106 disposed on the housing 102. The display 106 is electrically connected to the processor 104. The display 106 can be configured to present information related to functions performed by the weight scale 100, as described in more detail later.

The weight scale 100 includes a weight-bearing surface 108 that is configured to bear the weight of the subject. The weight-bearing surface 108 can be flexible or deformable to facilitate measurements as functions of such deformation. For example, the deformation can be measured using a strain gauge 110. The strain gauge 110 is disposed, for example, in the weight scale 100 beneath the weight-bearing surface 108. In some implementations, the strain gauge 110 includes a strain sensitive metal foil pattern 112 and two terminals 114a, 114b, which are electrically connected to the processor 104. The processor 104 causes an input voltage to be applied to the strain gauge 110. In some implementations, the processor 104 causes a power source to apply the input voltage to the strain gauge 110. When weight is applied to the weight scale 100, the weight-bearings surface 108 flexes and the strain sensitive metal foil pattern 112 temporarily deforms. The deformation causes the overall length of the strain sensitive metal foil pattern 112 to change, thereby altering the end-to-end resistance between the terminals 114a, and 114b. An output voltage between the terminals 114a, 114b corresponds to the change of resistance, and therefore is indicative of an amount of strain measured by the strain gauge 110. The strain measurement is then used for a number of purposes, as described in more detail below.

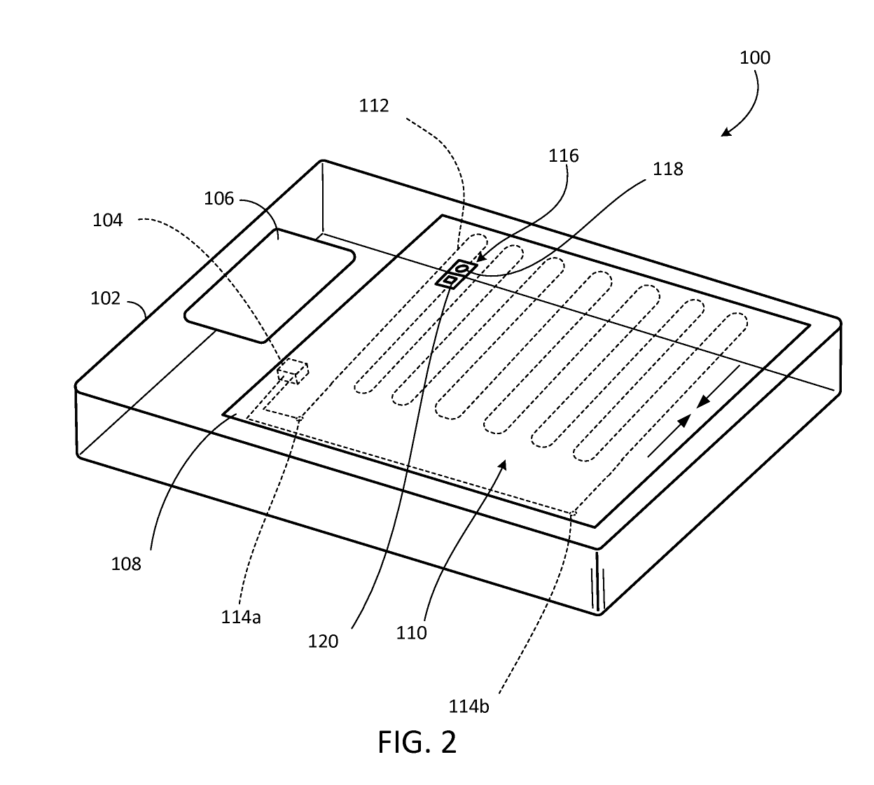

The strain gauge 110 utilizes the physical property of electrical conductance and its dependence on the geometry of the metal foil pattern 112 to measure an amount of strain. For example, referring briefly to FIG. 2, when a subject applies weight to the weight scale 100, the weight-bearing surface 108 flexes in a concave manner. The flexing causes a top surface of the strain sensitive metal foil pattern 112 to compress, thereby shortening the overall length and broadening the width of the strain sensitive metal foil pattern 112. When the overall length of the top surface of the strain sensitive metal foil pattern 112 is compressed and shortened, the end-to-end resistance between the terminals 114a, 114b is decreased. The processor 104 is configured to read an output voltage that corresponds to the change of resistance between the terminals 114a, 114b. The output voltage corresponds to the amount of strain measured by the strain gauge 110.

The strain measurements of the strain gauge 110 can be used for a number of purposes. For example, the strain gauge 110 of the weight scale 100 can be configured to measure the weight of the subject. The strain gauge 110 can be further configured to measure information about a motion of the subject including, for example, BCG (or MoCG) data. For example, when a subject is standing on the weight scale 100, BCG data can be measured at the subject's feet by capturing the mechanical reaction of the body due to blood flow at the feet.

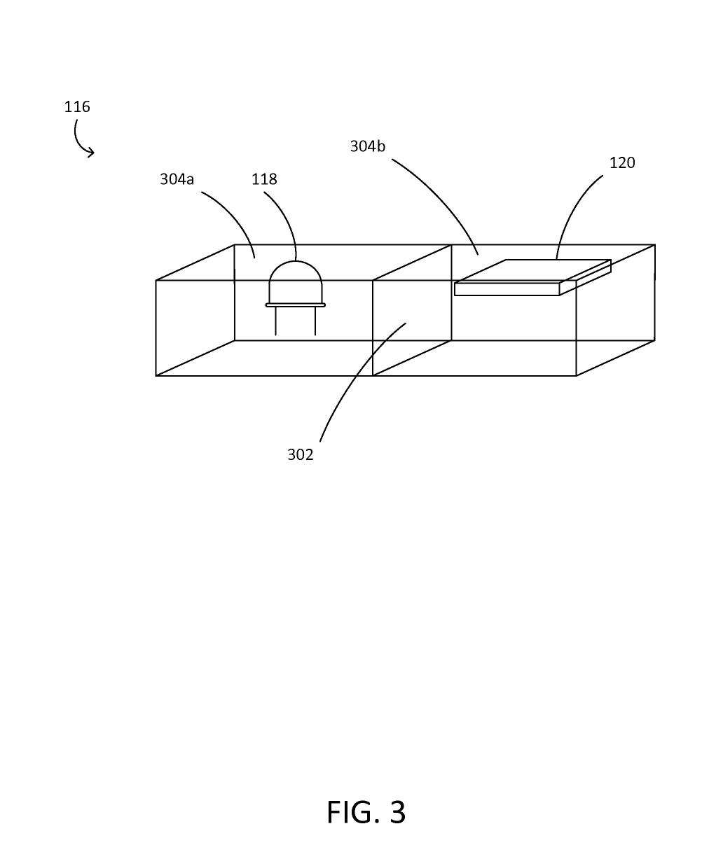

Referring to FIGS. 1 and 3, the weight scale 100 also includes a sensor insert 116 that is disposed in the weight-bearing surface 108. The sensor insert 116 includes a first compartment that houses an LED 118 and a second compartment that houses an optical sensor such as a photodiode 120. A divider or wall 302 separates the first compartment from the second compartment. The sensor insert 116 includes a first window 304a that is disposed above the first compartment and a second window 304b that is disposed above the second compartment. The windows 304a, 304b separate the LED 118 and the photodiode 120 from the exterior of the sensor insert 116. The windows 304a, 304b protect the LED 118 and the photodiode 120 from water, dirt, dust, and other debris. In some implementations, the windows 304a, 304b are made of acrylic.

The photodiode 120 is configured to measure information about pulse waves propagating through blood in the subject, such as PPG data. When the weight scale 100 is bearing the weight of the subject, the subject's feet make contact with the weight-bearing surface 108 and the sensor insert 116. In operation, light from the LED 118 is directed toward the skin on the bottom of the subject's foot, and the reflected light is measured using the photodiode 120. The reflected light is modulated by time-varying pulse waves within vasculature underneath the skin. Accordingly, an output signal from the photodiode represents the PPG. The photodiode 120 receives the reflected light and provides such an output signal to the processor 104. The PPG signal is synchronized with the heartbeat and can therefore be used to determine biometric parameters such as the subject's heart rate.

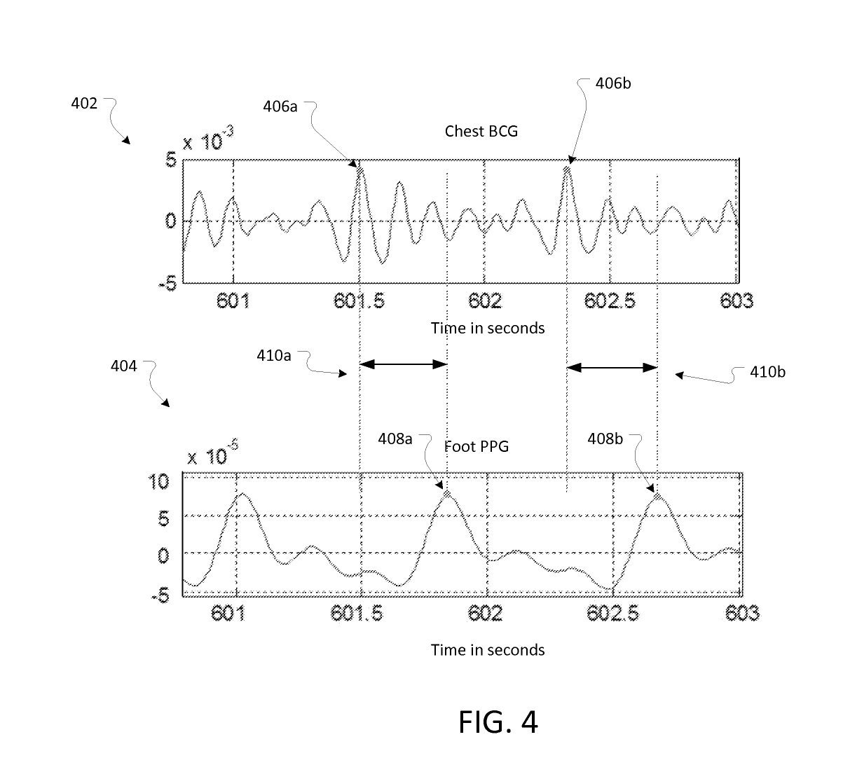

FIG. 4 illustrates calculation of PTTs using a BCG plot 402 and a PPG plot 404. The BCG plot 402 represents BCG data collected by the strain gauge 110, and the PPG plot 404 represents PPG data collected by the photodiode 120. In some implementations, the BCG data collected by the strain gauge 110 and the PPG data collected by the photodiode 120 can be used to calculate PTT, which can then be used to further calculate biometric parameters such as blood pressure, etc. In some implementations, the BCG data and the PPG data may be filtered prior to being used to calculate the PTT. The PTT can be calculated by determining the time difference between a first time point and a second time point at which a pulse wave through the vasculature traverses a first body part and a second body part, respectively. For example, the BCG plot 402 can be analyzed to determine the first time points, i.e., time points at which pulse waves originate at a given location of the subject's body. The PPG plot 404 can be analyzed to determine the second time points, i.e. corresponding time points at which the pulse waves arrive at another location of the subject's body. As such, the PTT represents the time it takes for a particular pulse wave to travel from one location of the subject's body to another location of the subject's body.

The BCG plot 402 includes reference points (e.g., local maxima) 406a, 406b that represent time points at which a corresponding pulse wave originates at a position near the subject's heart. These reference points 406a, 406b are referred to as pulse wave origination points 406. The PPG plot 404 also includes reference points (e.g., local maxima) 408a, 408b that represent time points at which a corresponding pulse wave arrives at the foot of the subject. These reference points 408a, 408b are referred to as pulse wave arrival points 408. The BCG plot 402 is time-aligned with the PPG plot 404 such that the PTT 410 between the position near the subject's heart and the foot can be determined as a time difference between the pulse wave origination points 406 at the position near the subject's heart and the corresponding pulse wave arrival points 408 at the foot. For example, the time difference between 406a and 408a represents the PTT 410a, and the time difference between 406b and 408b represents the PTT 410b.

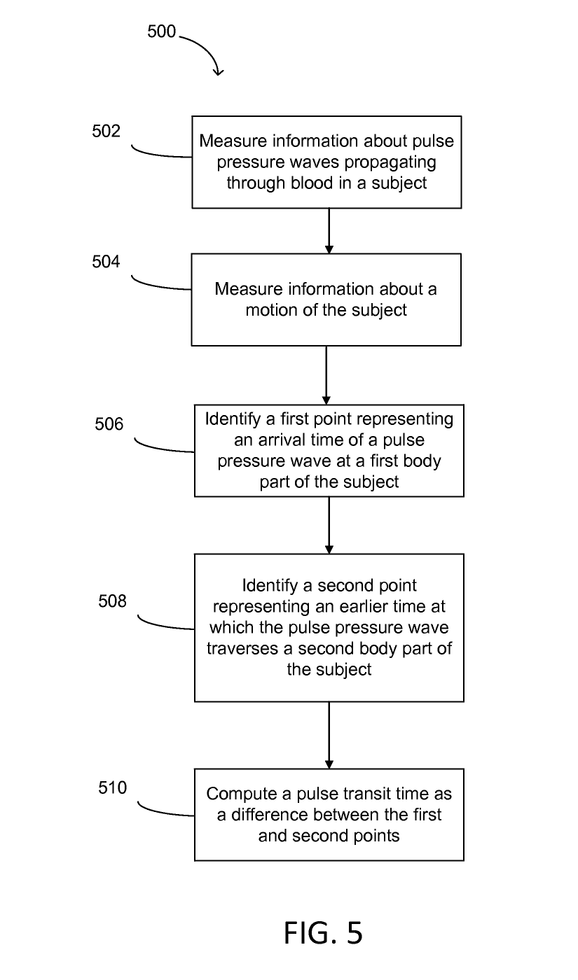

FIG. 5 shows a flowchart for an example process 500 of calculating a PTT. In some implementation, the process 500 is executed in the processor 104 of the weight scale 100 shown in FIG. 1. The process 500 includes measuring information about pulse waves propagating through blood in a subject (502). The information about pulse waves propagating through blood in the subject (e.g., PPG data) is measured by and received from the photodiode 120 disposed in the sensor insert 116. This can include, for example, directing light from the LED 118 toward the skin on the bottom of the subject's foot, and measuring the reflected light that is modulated by blood flow in the vasculature underneath the skin. Measuring the reflected light can include receiving the reflected light using the photodiode 120 and providing a resulting PPG signal dataset to the processor 104.

The process 500 also includes measuring information about a motion of the subject (504). The information about a motion of the subject (e.g., BCG data) can be measured, for example, using the strain gauge 110 disposed in the weight scale 100 beneath the weight-bearing surface 108.

The process 500 also includes identifying a first point representing an arrival time of a pulse wave at a first body part (e.g., a portion of a foot) of the subject (506). In some implementations, the first point can be identified from a PPG dataset. For example, identifying the first point can include identifying a reference point (e.g., a local maximum of the first derivative) within an expected range of time and/or amplitude of the PPG dataset.

The process 500 further includes identifying a second point representing an earlier time at which the pulse wave traverses a second body part (e.g., the chest) of the subject (508). In some implementations, the second point can be identified from a BCG dataset. For example, identifying the second point can include identifying a reference point (e.g., a local maximum) within an expected range of time and/or amplitude of the BCG dataset. The local maximum can be taken as a representation of the second point. The second point represents an earlier time at which the pulse wave originates at the position near the subject's heart.

The process 500 further includes computing a PTT as a difference between the first and second points (510). The PTT represents a time taken by the pulse wave to travel from the second body part to the first body part of the subject (e.g., from a portion of the chest proximate to the heart of the subject, through the vasculature, to the foot of the subject). The computed PTT can be used for calculating one or more health related parameters including, for example, systolic blood pressure and diastolic blood pressure.

While certain implementations have been described above, various other implementations are possible.

In some implementations, the sensor insert 116 can include one or more other light sources and/or one or more other optical sensors instead of or in addition to the LED 118 and the photodiode. Further, in some implementations, the windows 304a, 304b can be made of glass, plastic, polycarbonate, or any other suitable material.

In some implementations, a derivative of the PPG data collected by the photodiode 120 (represented as PPG plot 404 in FIG. 4) can be taken to more easily visualize the reference points 406a, 406b (shown in FIG. 4) that represent time points at which a corresponding pulse wave originates at the position near the subject's heart and the reference points 408a, 408b that represent time points at which a corresponding pulse wave arrives at the foot of the subject.

In some implementations, one or more of the reference points 406a, 406b of the BCG plot 402 and the reference points 408a, 408b of the PPG plot 404 can be local minima or zero-crossing points.

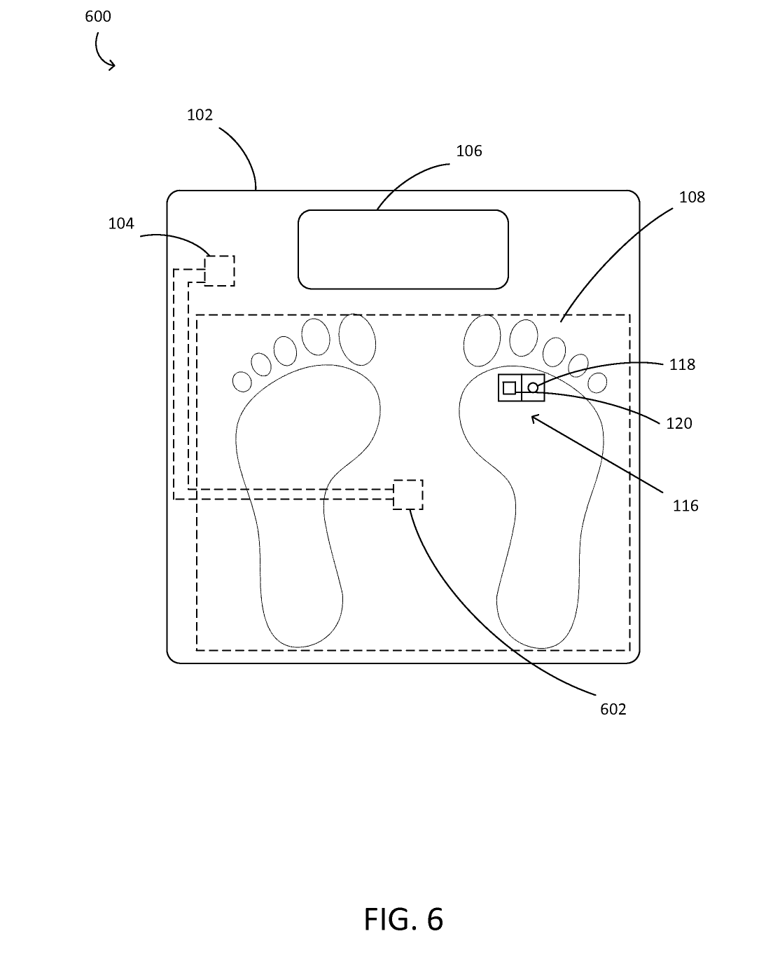

In some implementations, one or more other sensors can be disposed in the weight-bearing surface instead of or in addition to the strain gauge to measure the weight of the subject or to measure information about a motion of the subject. FIG. 6 shows an alternative implementation of a weight scale 600 that includes a motion sensor 602 disposed in the weight-bearing surface 108. At least a portion of the motion sensor 602 can be disposed beneath a plane defined by the weight-bearing surface 108. The motion sensor 602 is electrically connected to the processor 104.

In some implementations, the motion sensor 602 includes one or more accelerometers (e.g., one for each of the x, y and z axes). In some implementations, the motion sensor 602 can include one or more gyroscopes for measuring tilt, rotation, and yaw. The gyroscope can be configured to measure data that is used to refine the measurements from the accelerometer, thereby increasing the overall measurement accuracy of the motion sensor 602.

When the subject applies weight to the weight scale 600, the flexible weight-bearing surface 108 flexes in a concave manner. As a result, the motion sensor 602 moves. The processor 104 is configured to read an output from the motion sensor 602 that corresponds to the change of motion detected by the motion sensor 602. The change of motion measured by the motion sensor 602 can be used to measure the weight of the subject or to measure information about a motion of the subject, such as BCG data, which is measured relative to the vertical axis of the body. When the weight scale 600 is bearing the weight of the subject, the subject's feet make contact with the weight-bearing surface 108. The bottoms of the subject's feet experience a mechanical motion in response to the pulse waves. While the motion sensor 602 is already displaced due to the subject's weight, the pulsate motions cause the motion sensor 602 to be further displaced with each pulsate motion. That is, upon each pulsate motion, the weight-bearing surface 108 slightly further flexes in a concave manner. In between pulses, the weight-bearing surface returns to its flexed position that results from the subject's weight. The motion sensor 602 provides a resulting BCG signal to the processor 104 that corresponds to this periodic displacement.

Various points of the BCG signal are related to times at which pulse waves traverse a particular position of the subject's body. BCG data collected by the motions sensor 602 is analyzed in a similar fashion as the BCG data collected by the strain gauge 110 (shown in FIG. 1) that is included in other implementations of the weight-bearing device to determine times at which pulse waves originate at a given position of the subject's body (e.g., a position near the subject's heart). The PPG data collected by the photodiode 120 (shown in FIGS. 1 and 3) is analyzed to determine times at which the pulse waves arrive at another position of the subject's body (e.g., at the subject's foot). The determined times can be used to calculate the PTT.

In some implementations, the weight-bearing surface of the weight scale can be non-flexible. When a subject applies weight to the weight scale, the weight-bearing surface can resist flexing (e.g., in a concave manner).

In some implementations, such as when the weight-bearing surface of the weight scale is rigid, one or more pressure sensors, such as transducers, can be used instead of a strain gauge or a motion sensor. The pressure sensors can be disposed at locations on the weight-bearing surface where one or both of the subject's feet make contact. For example, a first pressure sensor can be disposed near or integrated into the sensor insert, and a second pressure sensor can be disposed opposite the first pressure sensor. The pressure sensors are electrically connected to the processor, and the processor is configured to read an output from the pressure sensors that corresponds to the pressure measured by the pressure sensors. The pressure measured by the pressure sensors can be used to measure the weight of the subject or to measure information about a motion of the subject in a similar way as described above with reference to the strain gauge 110 (shown in FIGS. 1 and 2) and the motion sensor 602 (shown in FIG. 6).

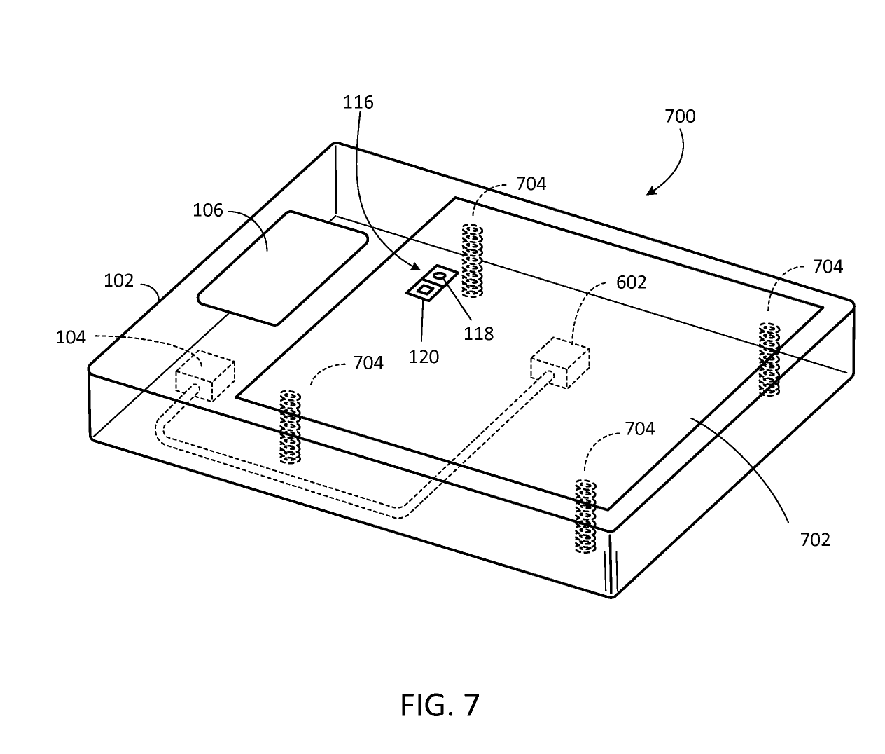

In some implementations, the motion sensor 602 can be included in an alternative implementation of the weight scale that has a non-flexible weight-bearing surface that is movably affixed to the housing 102 by a mechanism configured to permit the weight-bearing surface to depress. FIG. 7 shows an alternative implementation of a weight scale 700 that includes a motion sensor 602 disposed in a rigid weight-bearing surface 702 and springs 704 disposed between a bottom surface of the housing 102 and an underside of the weight-bearing surface 702. The motion sensor 602 is electrically connected to the processor 104.

When the subject applies weight to the weight scale 700, the weight-bearing surface 702 and the motion sensor 602 are vertically displaced. The processor 104 is configured to read an output from the motion sensor 602 that corresponds to the change of motion detected by the motion sensor 602. The change of motion measured by the motion sensor 602 can be used to measure the weight of the subject or to measure information about a motion of the subject in a similar way as described above with reference to FIG. 6.

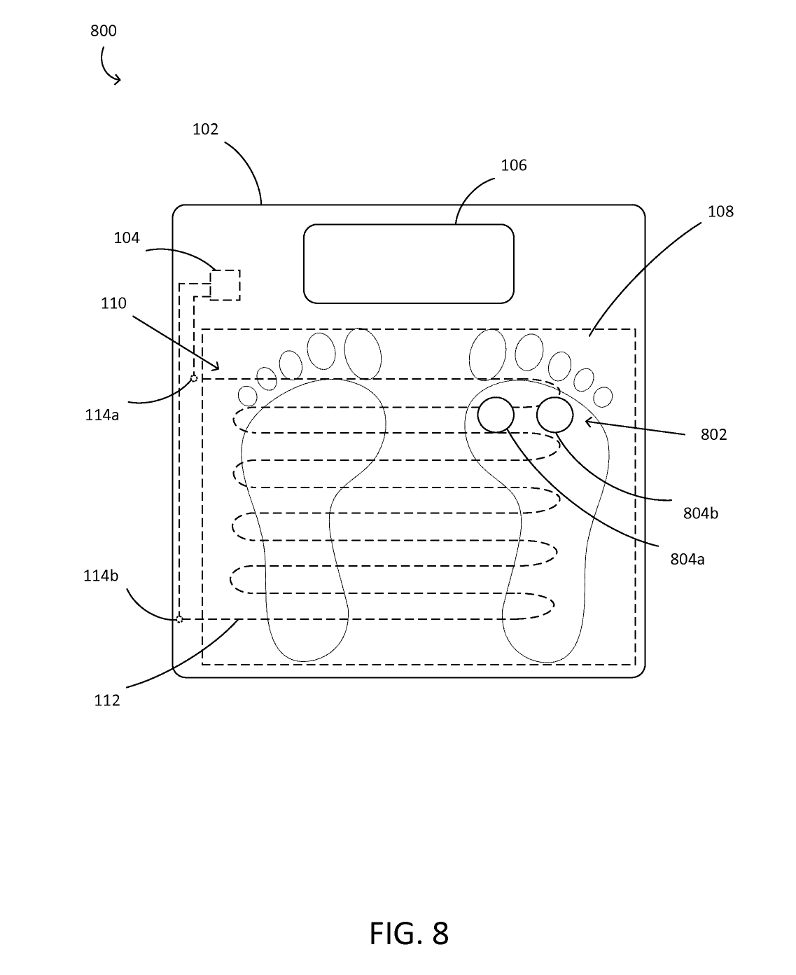

In some implementations, one or more other components and/or sensors can be disposed in the weight-bearing surface instead of or in addition to the LED and photodiode to measure information about pulse waves propagating through blood in the subject. FIG. 8 shows an alternative implementation of a weight scale 800 that includes an impedance sensor 802 disposed in the weight-bearing surface 108. The impedance sensor 802 includes two electrodes 804a, 804b that are positioned on the weight-bearing surface 108 such that a foot of the subject makes direct contact with both of the electrodes when the subject steps onto the weight scale 800. The electrodes can be positioned less than 4 inches (e.g., less than 3 inches, less than 2 inches, less than 1 inch, between 1 inch and 4 inches, etc.) of each other. The impedance sensor 802 is electrically connected to the processor 104.

The impedance sensor 802 is configured to obtain bio-impedance data of the subject. The electrodes 804a, 804b apply a voltage (e.g., of approximately 0.5-1.5 volts) across a particular portion of the subject's body (e.g., across a portion of the subject's foot) and measure the resultant current. The current seeks the path of least resistance, which is through the blood of the subject. The voltage and current values can be used to determine the impedance of the blood. The impedance is typically in the order of 10 k-100 k ohms. Time-varying changes in blood volume of the vasculature result in corresponding changes in the measured blood impedance. The impedance sensor 802 provides a resulting bio-impedance signal to the processor 104 that corresponds to these time-varying volumetric changes.

Each cardiac cycle is represented as a pattern of crests and troughs of a bio-impedance waveform, with each crest related to the arrival of a pulse wave at a particular position of the subject's body. As such, bio-impedance data can be used instead of PPG data to determine the arrival of a pulse wave at a particular position of the subject's body. BCG data is collected and analyzed in any of the ways described above to determine times at which pulse waves originate at a given position of the subject's body (e.g., a position near the subject's heart). The bio-impedance data collected by the impedance sensor 802 is analyzed in a similar fashion as the PPG data collected by the photodiode 120 (shown in FIGS. 1 and 3) that is included in other implementations of the weight scale to determine times at which the pulse waves arrive at another location of the subject's body (e.g., at the subject's foot). The determined times can be used to calculate PTT.

The impedance sensor 802 can also be configured to measure a body composition (e.g., a fat content) of the subject. In some implementations, the processor 104 can analyze the bio-impedance data obtained by the impedance sensor 802 as described above to determine the body composition of the subject. In some implementations, the impedance sensor 802 obtains additional data to determine the body composition of the subject. For example, the impedance sensor 802 and the processor 104 can utilize a technique such as a bioelectrical impedance analysis (BIA) to determine the fat content of the subject. The BIA can include causing a relatively small and harmless electrical current to be passed through a portion of the body of the subject, and measuring an electrical impedance encountered by the current. The current can be applied, for example, using the electrodes 804a, 804b, and the resultant voltage can be measured across the electrodes 804a, 804b. Current passes more easily through fat-free tissue like muscle than it does through fat or bone tissue. The values of the applied current and the measured voltage can be used to determine the impedance of the current path, and the impedance of the current path can be analyzed by the processor 104 to determine the composition of the current path (e.g., the body composition of the subject). For example, the magnitude of the impedance measurement can correspond to the fat content of the current path. A high impedance can therefore correspond to relatively high fat content, and a low impedance can correspond to relatively low fat content. The processor 104 can use additional information in determining the fat content that corresponds to the impedance measurement. For example, the processor 104 may use calibration data associated with one or more biological characteristics (e.g., height, weight, gender, age, etc.) in determining the fat content of the current path from the measured impedance.

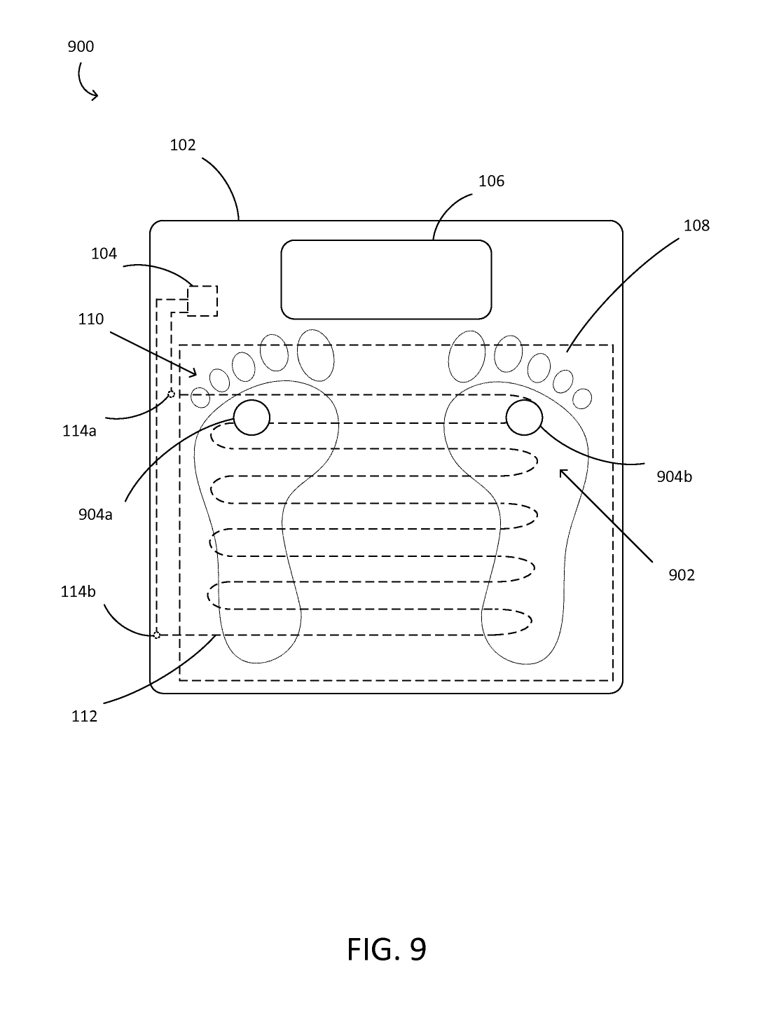

FIG. 9 shows an alternative implementation of a weight scale 900 that includes an impedance sensor 902 that includes two electrodes 904a, 904b that are positioned on the weight-bearing surface 108 such that a first foot of the subject makes direct contact with the first electrode 904a and a second foot of the subject makes direct contact with the second electrode 904b when the subject steps onto the weight scale 900. The electrodes can be positioned greater than or equal to 4 inches (e.g., greater than 5 inches, greater than 6 inches, greater than 7 inches, between 4 inches and 7 inches, etc.) from each other. The electrodes 904a, 904b apply a voltage across a portion of the subject's body (e.g., from one of the subject's feet to the other) and measure the resultant current. The current seeks the path of least resistance, which is through the blood of the subject. The voltage and current values can be used to determine the impedance of the blood. In this implementation, the impedance is measured through a relatively large portion of the subject's body, rather than, for example, through a relatively small portion of one of the subject's feet. As such, the bio-impedance data is used to determine the arrival of a pulse wave at a particular position of the subject's body that is somewhere other than the subject's foot. The particular position of the subject's body may be somewhere in the subject's torso or abdominal region.

In some implementations, rather than being a standalone device, the weight scale can be integrated into a floor. For example, the weight scale and its internal components can be a floor tile such as a ceramic tile. The floor tile can be integrated into a floor such as a bathroom floor, a kitchen floor, a shower floor, etc.

In some implementations, the weight-bearing biofeedback device can be any device configured to bear the weight of a subject. Such weight-bearing biofeedback devices can collect BCG and PPG data (and, instead of or in addition to the PPG data, bio-impedance data) and perform various biometric measurements based on the collected data.

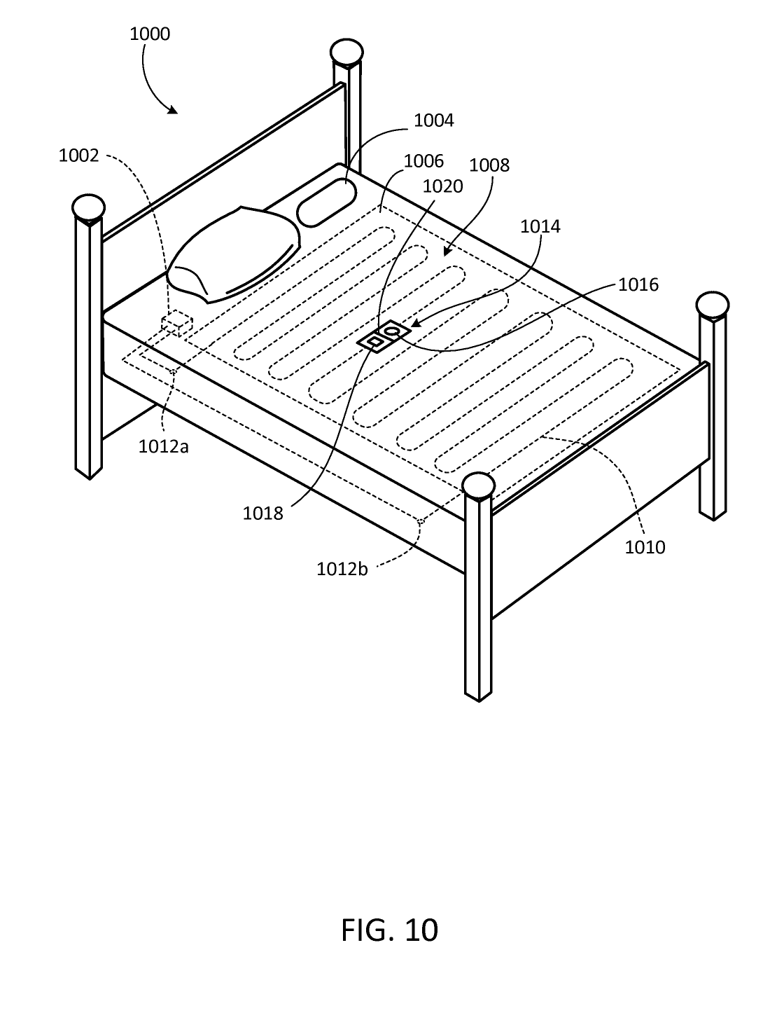

FIG. 10 shows a biofeedback bed 1000 that includes a processor 1002 and a display 1004 that is electrically connected to the processor 1002. The display 1004 is configured to present information related to functions performed by the biofeedback bed 1000.

The biofeedback bed 1000 includes a weight-bearing surface 1006 that is configured to bear the weight of the subject. The weight-bearing surface 1006 is flexible. A strain gauge 1008 is disposed in the biofeedback bed 1000 beneath the weight-bearing surface 1006. The strain gauge 1008 includes a strain sensitive metal foil pattern 1010 and two terminals 1012a, 1012b. The two terminals 1012a, 1012b are electrically connected to the processor 1002. The strain gauge 1008 operates in a similar fashion as the strain gauge 110 described with reference to FIGS. 1 and 2.

The strain measurements of the strain gauge 1008 can be used to measure the weight of the subject and also to measure information about a motion of the subject, such as MoCG data. BCG data and seismocardiogram (SCG) data are two examples of MoCG data. Both BCG and SCG are pulsatile motion signals of the body. While BCG is measured relative to the vertical axis of the body, SCG data is not limited to the vertical axis. SCG data represents cardiac vibrations as measured at a position of the subject's body (e.g., at the subject's back, chest, side, etc.) that is in contact with the weight-bearing surface 1006 of the biofeedback bed 1000. When the biofeedback bed 1000 is bearing the weight of the subject, the subject's back, chest, or side typically makes contact with the weight-bearing surface 1006. The subject experiences a mechanical motion in response to the pulse waves. These pulsate motions are measured by the strain gauge 1008, which provides a resulting MoCG signal to the processor 1002. The MoCG signal may include both a BCG signal and a SCG signal. In some implementations, the SCG signal dominates the BCG signal.

The biofeedback bed 1000 also includes a sensor insert 1014 (substantially similar to the sensor insert 116 described with reference to FIG. 3) that is disposed in the weight-bearing surface 1006. The sensor insert 1014 includes a first compartment that houses an LED 1016, a second compartment that houses an optical sensor such as a photodiode 1018, a wall 1020 that separates the first compartment from the second compartment, a first window that is disposed above the first compartment, and a second window that is disposed above the second compartment.

The photodiode 1018 is configured to measure information about pulse waves propagating through blood in the subject, such as PPG data, in a similar fashion as the photodiode 120 described with reference to FIGS. 1 and 3. When the biofeedback bed 1000 is bearing the weight of the subject, a portion of the subject's body (e.g., a portion of the subject's back, chest, arms, legs, torso, etc.) makes contact with the sensor insert 1014. In operation, light from the LED 1016 is directed toward the skin of the subject, and the reflected light is modulated by blood flow underneath the skin. The photodiode 1018 receives the reflected light and provides a resulting signal to the processor 1002. The light emitted from the LED 1016 can be an invisible wavelength light so as not to disturb the subject's sleep.

The MoCG data collected by the strain gauge 1008 and the PPG data collected by the photodiode 1018 can be used to calculate PTT, which can be used to further calculate the biometric parameters. The MoCG data is analyzed to determine times at which pulse waves originate at a given position of the subject's body (e.g., at a position near the subject's heart), and the PPG data is analyzed to determine times at which the pulse waves arrive at another position of the subject's body (e.g., at a portion of the subject's torso). The differences between these times represent the PTT.

In some implementations, the biofeedback bed 1000 can include an impedance sensor disposed in the weight-bearing surface 1006. The impedance sensor includes two electrodes that are positioned on the weight-bearing surface 1006 such that a part of the skin of the subject makes direct contact with both of the electrodes when the biofeedback bed 1000 bears the weight of the subject.

The impedance sensor is configured to obtain bio-impedance data of the subject. The electrodes apply a voltage across a particular portion of the subject's body and measure the resultant current. The voltage and current values can be used to determine the impedance of the blood. Time-varying changes in blood volume of the vasculature result in corresponding changes in the measured blood impedance. The impedance sensor provides a resulting bio-impedance signal to the processor 1002 that corresponds to these time-varying volumetric changes.

As described above, bio-impedance data can be used instead of PPG data to determine the arrival of a pulse wave at a particular position of the subject's body. MoCG data collected by the strain gauge 1008 is analyzed as described above to determine times at which pulse waves originate at a given location of the subject's body (e.g., a position near the subject's heart). The bio-impedance data collected by the impedance sensor is analyzed in a similar fashion as the PPG data collected by the photodiode 1018 to determine times at which the pulse waves arrive at another position of the subject's body (e.g., at a portion of the subject's torso). The determined times can be used to calculate PTT, which represents the time it takes for a pulse wave to travel from one position of the subject's body to another position of the subject's body.

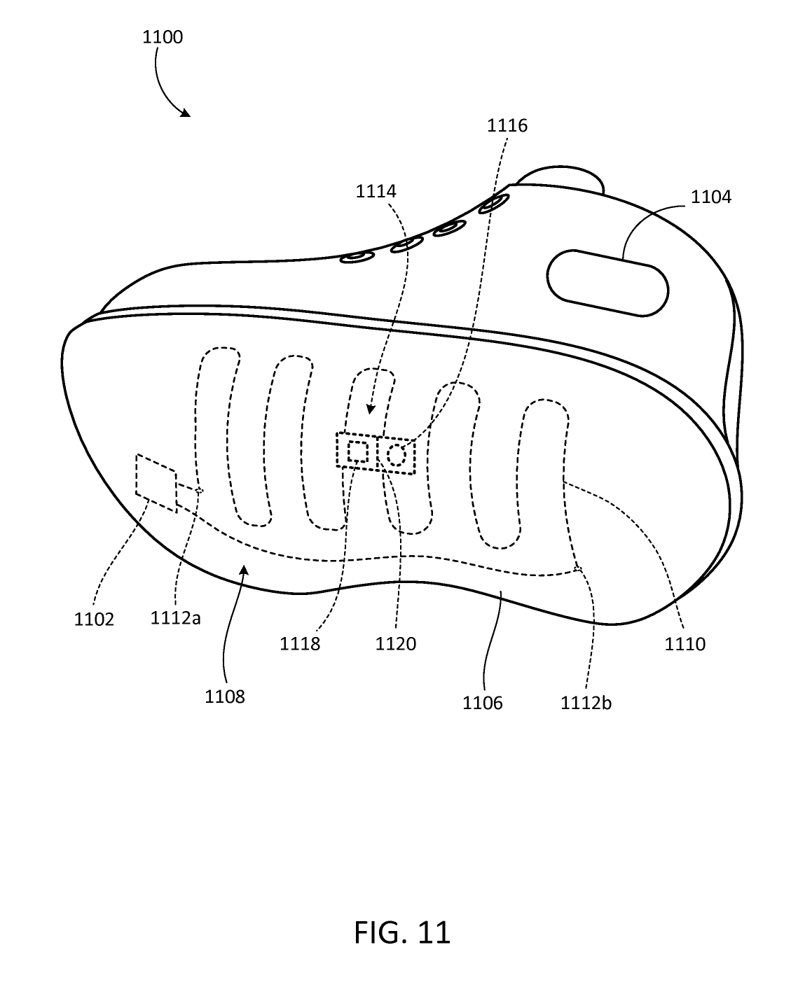

FIG. 11 shows a biofeedback shoe 1100 that includes a processor 1102 and a display 1104 that is electrically connected to the processor 1102. The display 1104 is configured to present information related to functions performed by the biofeedback shoe 1100.

The biofeedback shoe 1100 includes a weight-bearing surface 1106 that is, e.g., a sole of the biofeedback shoe 1100. The weight-bearing surface 1106 is flexible and is configured to bear the weight of the subject. A strain gauge 1108 is disposed in the weight-bearing surface 1106 of the biofeedback shoe 1100. The strain gauge 1108 includes a strain sensitive metal foil pattern 1110 and two terminals 1112a, 1112b. The two terminals 1112a, 1112b are electrically connected to the processor 1102. The strain gauge 1108 operates in a similar fashion as the strain gauge 110 described with reference to FIGS. 1 and 2.

The strain measurements of the strain gauge 1108 can be used to measure the weight of the subject and also to measure information about a motion of the subject, such as BCG data. When the biofeedback shoe 1100 is bearing the weight of the subject, the subject's foot makes contact with the weight-bearing surface 1106. The bottom of the subject's foot experiences a mechanical motion in response to the pulse waves. These pulsate motions are measured by the strain gauge 1108, which provides a resulting BCG signal to the processor 1102.

The biofeedback shoe 1100 also includes a sensor insert 1114 (substantially similar to the sensor insert 116 described with reference to FIG. 3) that is disposed in the weight-bearing surface 1106. The sensor insert 1114 includes a first compartment that houses an LED 1116, a second compartment that houses an optical sensor such as a photodiode 1118, a wall 1120 that separates the first compartment from the second compartment, a first window that is disposed above the first compartment, and a second window that is disposed above the second compartment.

The photodiode 1118 is configured to measure information about pulse waves propagating through blood in the subject, such as PPG data, in a similar fashion as the photodiode 120 described with reference to FIGS. 1 and 3. When the biofeedback shoe 1100 is bearing the weight of the subject, the subject's foot makes contact with the sensor insert 1114. In operation, light from the LED 1116 is directed toward the skin of the subject, and the reflected light is modulated by blood flow underneath the skin. The photodiode 1118 receives the reflected light and provides a resulting signal to the processor 1102.

The BCG data collected by the strain gauge 1108 and the PPG data collected by the photodiode 1118 can be used to calculate PTT.

In some implementations, the biofeedback shoe 1100 can include an impedance sensor disposed in the weight-bearing surface 1106. The impedance sensor includes two electrodes that are positioned on the weight-bearing surface 1106 such that the foot of the subject makes direct contact with both of the electrodes when the biofeedback shoe 1100 bears the weight of the subject. Bio-impedance data can be used instead of PPG data to determine the arrival of a pulse wave at the foot of the subject.

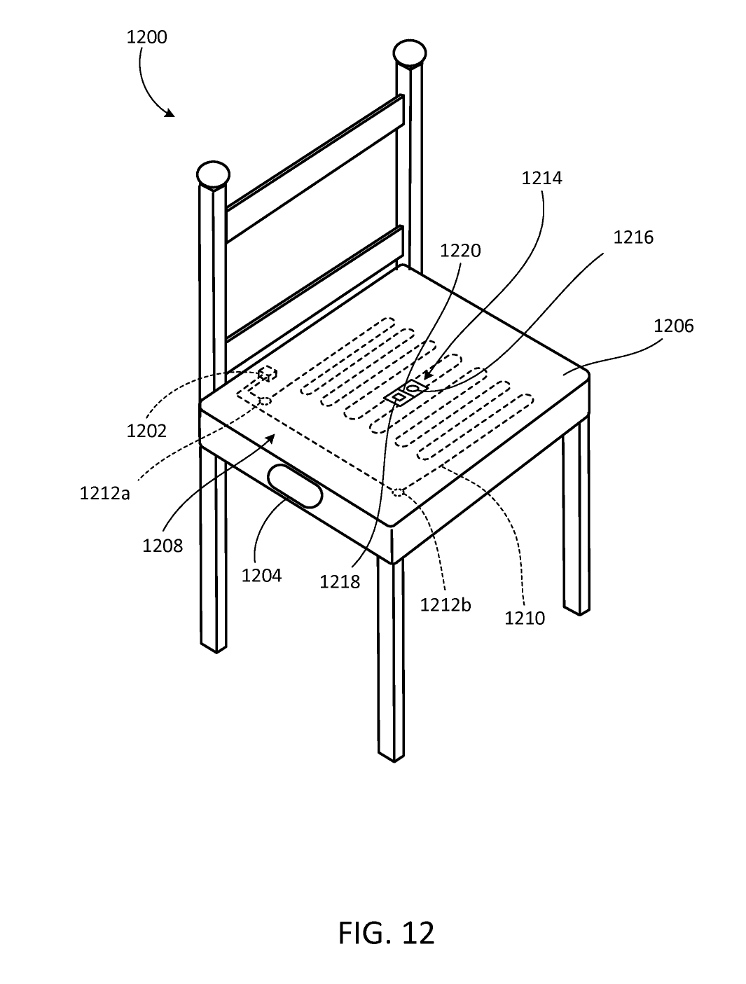

FIG. 12 shows a biofeedback chair 1200 that includes a processor 1202 and a display 1204 that is electrically connected to the processor 1202. The display 1204 is configured to present information related to functions performed by the biofeedback chair 1200.

The biofeedback chair 1200 includes a weight-bearing surface 1206, e.g., a cushion of the biofeedback chair 1200. The weight-bearing surface 1206 is flexible and is configured to bear the weight of the subject. A strain gauge 1208 is disposed in the weight-bearing surface 1206 of the biofeedback chair 1200. The strain gauge 1208 includes a strain sensitive metal foil pattern 1210 and two terminals 1212a, 1212b. The two terminals 1212a, 1212b are electrically connected to the processor 1202. The strain gauge 1208 operates in a similar fashion as the strain gauge 110 described with reference to FIGS. 1 and 2.

The strain measurements of the strain gauge 1208 can be used to measure the weight of the subject and also to measure information about a motion of the subject, such as BCG data. When the biofeedback chair 1200 is bearing the weight of the subject, the subject's backside makes contact with the weight-bearing surface 1206. The subject's bottom (e.g., buttocks) experiences a mechanical motion in response to the pulse waves. These pulsate motions are measured by the strain gauge 1208, which provides a resulting BCG signal to the processor 1202.

The biofeedback chair 1200 also includes a sensor insert 1214 (substantially similar to the sensor insert 116 described with reference to FIG. 3) that is disposed in the weight-bearing surface 1206. The sensor insert 1214 includes a first compartment that houses an LED 1216, a second compartment that houses an optical sensor such as a photodiode 1218, a wall 1220 that separates the first compartment from the second compartment, a first window that is disposed above the first compartment, and a second window that is disposed above the second compartment.

The photodiode 1218 is configured to measure information about pulse waves propagating through blood in the subject, such as PPG data, in a similar fashion as the photodiode 120 described with reference to FIGS. 1 and 3. When the biofeedback chair 1200 is bearing the weight of the subject, the subject's backside makes contact with the sensor insert 1214. In operation, light from the LED 1216 is directed toward the skin of the subject, and the reflected light is modulated by blood flow underneath the skin. The photodiode 1218 receives the reflected light and provides a resulting signal to the processor 1202.

The BCG data collected by the strain gauge 1208 and the PPG data collected by the photodiode 1218 can be used to calculate PTT.

In some implementations, the biofeedback chair 1200 can include an impedance sensor disposed in the weight-bearing surface 1206. The impedance sensor includes two electrodes that are positioned on the weight-bearing surface 1206 such that the underside of the subject makes direct contact with both of the electrodes when the biofeedback chair 1200 bears the weight of the subject. Bio-impedance data can be used instead of PPG data to determine the arrival of a pulse wave at the backside of the subject.

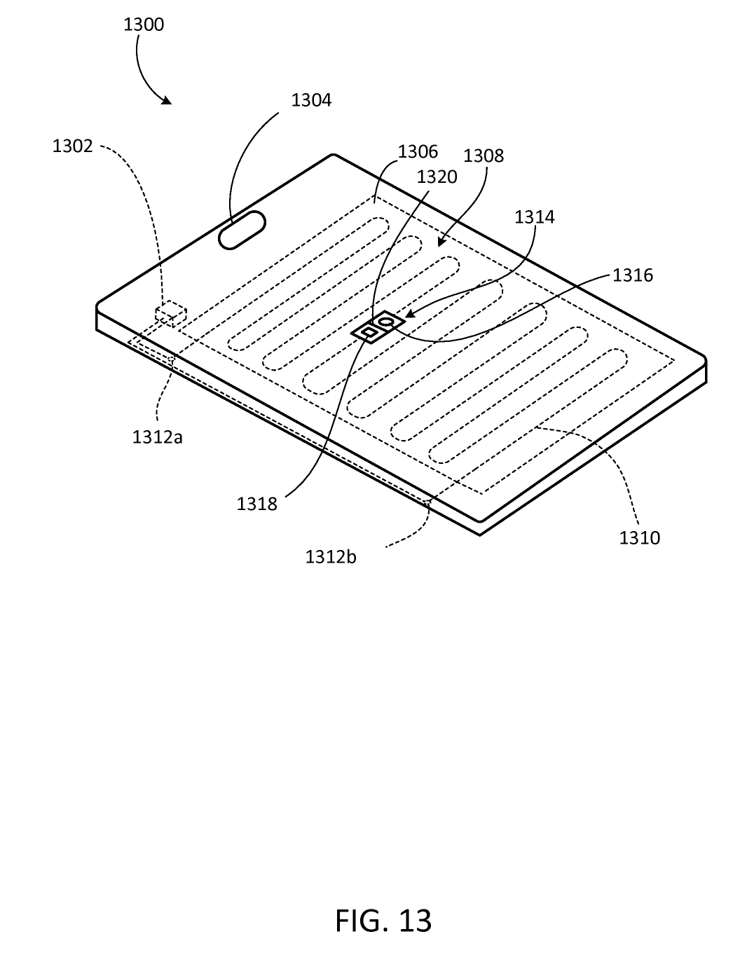

FIG. 13 shows a biofeedback yoga mat 1300 that includes a processor 1302 and a display 1304 that is electrically connected to the processor 1302. The display 1304 is configured to present information related to functions performed by the biofeedback yoga mat 1300.

The biofeedback yoga mat 1300 includes a weight-bearing surface 1306 that is configured to bear the weight of the subject (e.g., while the subject is performing yoga). The weight-bearing surface 1306 is flexible. A strain gauge 1308 is disposed in the biofeedback yoga mat 1300 beneath the weight-bearing surface 1306. The strain gauge 1308 includes a strain sensitive metal foil pattern 1310 and two terminals 1312a, 1312b. The two terminals 1312a, 1312b are electrically connected to the processor 1302. The strain gauge 1308 operates in a similar fashion as the strain gauge 110 described with reference to FIGS. 1 and 2.

The strain measurements of the strain gauge 1308 can be used to measure the weight of the subject and also to measure information about a motion of the subject, such as MoCG data, including SCG data and BCG data. When the biofeedback yoga mat 1300 is bearing the weight of the subject, the subject's feet, backside, back, chest, or side typically makes contact with the weight-bearing surface 1306. The subject experiences a mechanical motion in response to the pulse waves. These pulsate motions are measured by the strain gauge 1308, which provides a resulting MoCG signal to the processor 1302. The MoCG signal may include both a BCG signal and a SCG signal. In some implementations, the SCG signal dominates the BCG signal.

The biofeedback yoga mat 1300 also includes a sensor insert 1314 (substantially similar to the sensor insert 116 described with reference to FIG. 3) that is disposed in the weight-bearing surface 1306. The sensor insert 1314 includes a first compartment that houses an LED 1316, a second compartment that houses an optical sensor such as a photodiode 1318, a wall 1320 that separates the first compartment from the second compartment, a first window that is disposed above the first compartment, and a second window that is disposed above the second compartment.

The photodiode 1318 is configured to measure information about pulse waves propagating through blood in the subject, such as PPG data, in a similar fashion as the photodiode 120 described with reference to FIGS. 1 and 3. When the biofeedback yoga mat 1300 is bearing the weight of the subject, a portion of the subject's body (e.g., a portion of the subject's feet, backside, back, chest, arms, legs, torso, etc.) makes contact with the sensor insert 1314. In operation, light from the LED 1316 is directed toward the skin of the subject, and the reflected light is modulated by blood flow underneath the skin. The photodiode 1318 receives the reflected light and provides a resulting signal to the processor 1302. The light emitted from the LED 1316 can be an invisible wavelength light so as not to disturb the subject during yoga.

The MoCG data collected by the strain gauge 1308 and the PPG data collected by the photodiode 1318 can be used to calculate PTT.

In some implementations, the biofeedback yoga mat 1300 can include an impedance sensor disposed in the weight-bearing surface 1306. The impedance sensor includes two electrodes that are positioned on the weight-bearing surface 1306 such that a part of the skin of the subject makes direct contact with both of the electrodes when the biofeedback yoga mat 1300 bears the weight of the subject.

The impedance sensor is configured to obtain bio-impedance data of the subject. The electrodes apply a voltage across a particular portion of the subject's body and measure the resultant current. The voltage and current values are used to determine the impedance of the blood. Time-varying changes in blood volume of the vasculature result in corresponding changes in the measured blood impedance. The impedance sensor provides a resulting bio-impedance signal to the processor 1302 that corresponds to these time-varying volumetric changes.

As described above, bio-impedance data can be used instead of PPG data to determine the arrival of a pulse wave at a particular position of the subject's body. MoCG data collected by the strain gauge 1308 is analyzed as described above to determine times at which pulse waves originate at a given position of the subject's body (e.g., a position near the subject's heart). The bio-impedance data collected by the impedance sensor is analyzed in a similar fashion as the PPG data collected by the photodiode 1318 to determine times at which the pulse waves arrive at another position of the subject's body (e.g., at a portion of the subject's torso, backside, or feet). The determined times are used to calculate PTT.

In some implementations, the biofeedback bed, the biofeedback shoe, the biofeedback chair, and/or the biofeedback yoga mat can include one or more other sensors instead of or in addition to the strain gauge to measure the weight of the subject or to measure information about a motion of the subject. For example, a motion sensor can be disposed in the weight-bearing surface. When the subject applies weight to the weight-bearing device, the flexible weight-bearing surface flexes in a concave manner. As a result, the motion sensor moves. The processor is configured to read an output from the motion sensor that corresponds to the change of motion detected by the motion sensor. The change of motion measured by the motion sensor can be used to measure the weight of the subject or to measure information about a motion of the subject.

In some implementations, the biofeedback bed, the biofeedback shoe, the biofeedback chair, and/or the biofeedback yoga mat can include a weight-bearing surface that is non-flexible. When a subject applies weight to the weight scale, the weight-bearing surface can resist flexing (e.g., in a concave manner).

In some implementations, such as when the weight-bearing surface is rigid, one or more pressure sensors, such as transducers, can be used instead of a strain gauge or a motion sensor. The pressure sensors can be disposed at locations on the weight-bearing surface where the subject's body makes contact. The pressure sensors are electrically connected to the processor, and the processor is configured to read an output from the pressure sensors that corresponds to the pressure measured by the pressure sensors. The pressure measured by the pressure sensors can be used to measure the weight of the subject or to measure information about a motion of the subject in a similar way as described above with reference to the strain gauge and the motion sensor.

In some implementations of the biofeedback bed, the biofeedback shoe, the biofeedback chair, and the biofeedback yoga mat, the motion sensor can be disposed in a non-flexible weight-bearing surface that is movably affixed to the weight-bearing biofeedback device by a mechanism configured to permit the weight-bearing surface to depress. For example, one or more springs can be disposed beneath an underside of the weight-bearing surface.

When the subject applies weight to the biofeedback bed, the biofeedback shoe, the biofeedback chair, or the biofeedback yoga mat, the weight-bearing surface and the motion sensor are vertically displaced. The processor is configured to read an output from the motion sensor that corresponds to the change of motion detected by the motion sensor. The change of motion measured by the motion sensor can be used to measure the weight of the subject or to measure information about a motion of the subject.

In some implementations, the PTT can be computed as a difference between two points included in time-varying information about at least one pulse wave propagating through blood in the subject. For example, the PTT can be computed as a difference between a first point in PPG data or bio-impedance data and a second point in PPG data or bio-impedance data.

In some implementations, the sensor insert can include any number of compartments and any number of LEDs and/or optical sensors. In such implementations, the LEDs are separated from the optical sensors by one or more walls of the sensor insert. In some implementations, the sensor insert includes a first compartment that houses a first LED, a second compartment adjacent to the first compartment that houses an optical sensor, and a third compartment adjacent to the second compartment that houses a second LED. A first wall separates the first compartment from the second compartment, and a second wall separates the second compartment from the third compartment.

In some implementations, the strain gauge is configured such that when the weight-bearing surface flexes in a concave manner, a bottom surface of the strain sensitive metal foil pattern stretches, thereby increasing the overall length and narrowing the width of the strain sensitive metal foil pattern. The terminals can be configured to respond to the deformation of the bottom surface of the strain sensitive metal foil pattern. When the overall length of the bottom surface of the strain sensitive metal foil pattern is stretched and lengthened, the end-to-end resistance between the terminals is increased. The processor is configured to read an output voltage that corresponds to the change of resistance between the terminals. The output voltage corresponds to the amount of strain measured by the strain gauge.

In some implementations, a current running through the strain sensitive metal foil pattern is measured to determine the end-to-end resistance between the terminals. In some implementations, the weight-bearing biofeedback device does not include one or more of the components described above. For example, in some implementations, the weight-bearing biofeedback device does not include a display.

In some implementations, the weight-bearing biofeedback device includes at least two LEDs and at least two accompanying photodiodes. Each photodiode is configured to measure information about pulse waves propagating through blood in the subject, such as PPG data. Each photodiode is positioned at a different location on the weight-bearing biofeedback device such that a first body part of the subject makes contact with the first photodiode and a second body part of the subject makes contact with the second photodiode. In operation, light from each LED is directed toward the skin at the respective body part of the subject, and the reflected light is modulated by blood flow underneath the skin. Each photodiode receives the reflected light and provides a resulting signal to the processor. Each photodiode produces a set of PPG data. The two sets of PPG data are used to calculate the PTT.

The first set of PPG data is analyzed to determine times at which the pulse waves arrive at the first body part of the subject, and the second set of PPG data is analyzed to determine times at which the pulse waves arrive at the second body part of the subject. Each set of PPG data includes reference points (e.g., local maxima) that represent time points at which a corresponding pulse wave arrives at the respective body part of the subject.

The PPG data plots are synchronized such that the PTT between the first body part of the subject and the second body part of the subject can be determined as a time difference between a reference point in the first set of PPG data that corresponds to the first body part and a reference point in the second set of PPG data that corresponds to the second body part.

In some implementations, the weight-bearing biofeedback device includes at least two impedance sensors that each includes two electrodes. Each impedance sensor is configured to measure information about pulse waves propagating through blood in the subject, such as bio-impedance data. Each impedance sensor is positioned at a different location on the weight-bearing biofeedback device such that a first body part of the subject makes contact with the first photodiode and a second body part of the subject makes contact with the second photodiode. Each pair of electrodes applies a voltage across the respective body part of the subject and measures the resultant current. The current seeks the path of least resistance, which is through the blood of the subject. The voltage and current values are used to determine the impedance of the blood. Each impedance sensor provides a resulting signal to the processor. Each impedance sensor produces a set of bio-impedance data. The two sets of bio-impedance data are used to calculate the PTT.

The first set of bio-impedance data is analyzed to determine times at which the pulse waves arrive at the first body part of the subject, and the second set of bio-impedance data is analyzed to determine times at which the pulse waves arrive at the second body part of the subject. Each set of bio-impedance data includes reference points (e.g., local maxima) that represent time points at which a corresponding pulse wave arrives at the respective body part of the subject.

The bio-impedance data plots are synchronized such that the PTT between the first body part of the subject and the second body part of the subject can be determined as a time difference between a reference point in the first set of bio-impedance data that corresponds to the first body part and a reference point in the second set of bio-impedance data that corresponds to the second body part.

In some implementations, the two LEDs and photodiodes are configured to produce a single set of PPG data. For example, the PPG data from the two photodiodes is averaged and used to produce one set of PPG data. The PPG data can then be used with MoCG data to calculate the PTT. Similarly, in some implementations, the two impedance sensors are configured to produce a single set of bio-impedance data. For example, the bio-impedance data from the two impedance sensors is averaged and used to produce one set of bio-impedance data. The bio-impedance data can then be used with MoCG data to calculate the PTT.

In some implementations, the weight-bearing biofeedback device includes an LED and an accompanying photodiode, as well as an impedance sensor that includes two electrodes. The photodiode and the impedance sensor are each configured to measure information about pulse waves propagating through blood in the subject, such as PPG data. The photodiode is positioned at a first location on the weight-bearing biofeedback device such that a first body part of the subject makes contact with the photodiode, and the impedance sensor is positioned at a second location on the weight-bearing biofeedback device such that a second body part of the subject makes contact with the impedance sensor. The photodiode produces a set of PPG data, and the impedance sensor produces a set of bio-impedance data. The set of PPG data and the set of bio-impedance data are used to calculate the PTT.

The set of PPG data is analyzed to determine times at which the pulse waves arrive at the first body part of the subject, and the set of bio-impedance data is analyzed to determine times at which the pulse waves arrive at the second body part of the subject. The set of PPG data and the set of bio-impedance data each includes reference points (e.g., local maxima) that represent time points at which a corresponding pulse wave arrives at the respective body part of the subject.

The PPG data plot and the bio-impedance data plot are synchronized such that the PTT between the first body part of the subject and the second body part of the subject can be determined as a time difference between a reference point in the set of PPG data that corresponds to the first body part and a reference point in the set of bio-impedance data that corresponds to the second body part.

While the various sensors of the weight-bearing biofeedback device have been described as being disposed at particular locations on the device, then sensors can alternatively be disposed at other locations. In some implementations, the sensor insert and/or the impedance sensor of the biofeedback shoe can be disposed in a side surface of the shoe such that the sensor insert and/or impedance sensor makes contact with the subject's ankle when the shoe is being worn. In some implementations, the sensor insert and/or the impedance sensor of the biofeedback chair can be disposed in a side of the chair such that the sensor insert and/or impedance sensor makes contact with the subject's thighs when the subject is sitting in the biofeedback chair.

In some implementations, the PTT can be approximated using electrocardiogram (ECG) data and PPG or bio-impedance data. An ECG is the measure of the electrical signals from the heart that are caused when the heart depolarizes. However, at a given depolarization, pressure builds up in the heart for some amount of time before blood is actually ejected. Thus, the ECG data is used as an approximate of the time when blood is ejected from the heart. As described above, the PTT is the actual time it takes for a pulse wave to travel from a first position of the subject's body (e.g., a position near the subject's heart) to a second position of the subject's body (e.g., the subject's foot). In contrast, the time difference between the time when the heart depolarizes and the time when the pulse wave arrives at the second position of the subject's body is referred to as the Pulse Arrival Time (PAT). Thus, the PAT is calculated using ECG data and PPG or bio-impedance data. The PAT is an approximation of the PTT.

Computing Device

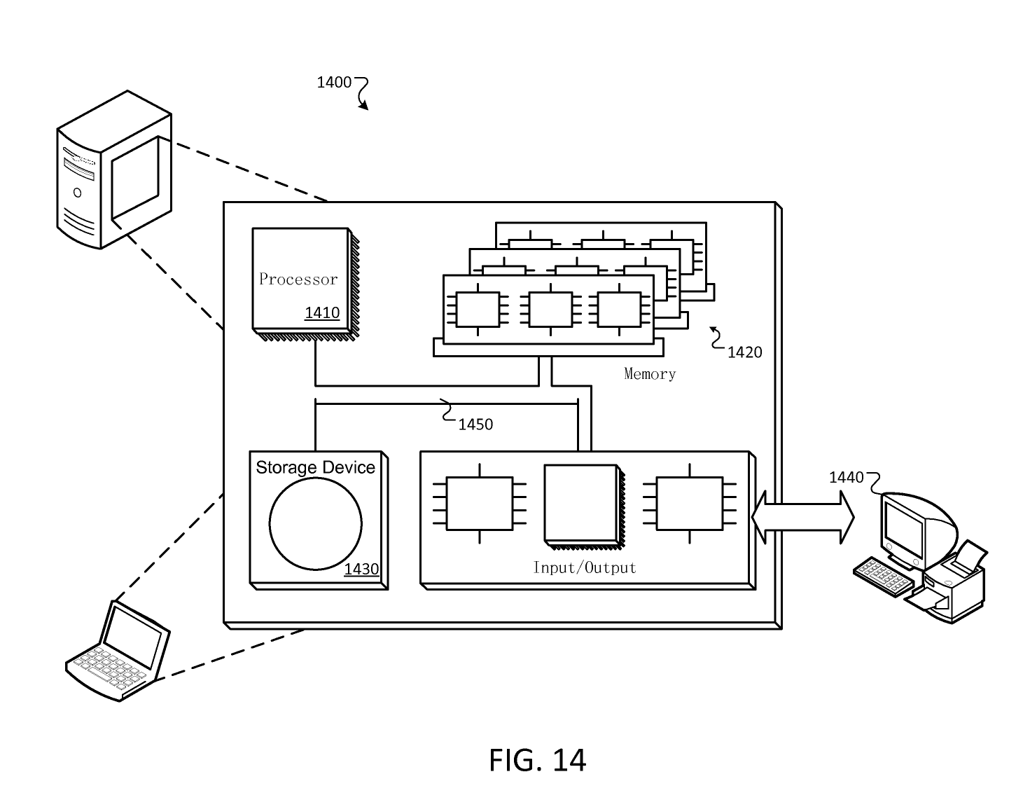

FIG. 14 is block diagram of an example computer system 1400 that can be used for performing one or more operations related to the technology described above. In some implementations, the computer system 1400 can be used to implement any portion, module, unit or subunit of the weight-bearing biofeedback device, or computing devices and processors referenced above. The system 1400 includes a processor 1410, a memory 1420, a storage device 1430, and an input/output device 1440. Each of the components 1410, 1420, 1430, and 1440 can be interconnected, for example, using a system bus 1450. The processor 1410 is capable of processing instructions for execution within the system 1400. In one implementation, the processor 1410 is a single-threaded processor. In another implementation, the processor 1410 is a multi-threaded processor. The processor 1410 is capable of processing instructions stored in the memory 1420 or on the storage device 1430.

The memory 1420 stores information within the system 1400. In one implementation, the memory 1420 is a computer-readable storage device that includes a non-transitory computer readable medium. In general, non-transitory computer readable medium is a tangible storage medium for storing computer readable instructions and/or data. In some cases, the storage medium can be configured such that stored instructions or data are erased or replaced by new instructions and/or data. Examples of such non-transitory computer readable medium include a hard disk, solid-state storage device, magnetic memory or an optical disk. In one implementation, the memory 1420 is a volatile memory unit. In another implementation, the memory 1420 is a non-volatile memory unit.

The storage device 1430 is capable of providing mass storage for the system 1400. In one implementation, the storage device 1430 is a computer-readable medium. In various different implementations, the storage device 1430 can include, for example, a hard disk device, an optical disk device, or some other large capacity storage device.

The input/output device 1440 provides input/output operations for the system 1400. In one implementation, the input/output device 1440 can include one or more of a network interface devices, e.g., an Ethernet card, a serial communication device, e.g., an RS-232 port, and/or a wireless interface device, e.g., and 802.11 card. In another implementation, the input/output device can include driver devices configured to receive input data and send output data to other input/output devices, e.g., keyboard, printer and display devices. In some implementations, the input/output device is configured to communicate with a network device such as a hub (e.g., an Ethernet hub) to facilitate communications between the computer system 1400 and other devices (e.g., other computer systems, a server, a network, etc.). In some implementations, the input/output device is configured to wirelessly communicate with a cloud-based network (e.g., to facilitate the storage of information on a remote server or a distributed computing system).

Although an example processing system has been described in FIG. 14, implementations of the subject matter and the functional operations described in this specification can be implemented in other types of digital electronic circuitry, or in computer software, firmware, or hardware, including the structures disclosed in this specification and their structural equivalents, or in combinations of one or more of them. Implementations of the subject matter described in this specification can be implemented as one or more computer program products, i.e., one or more modules of computer program instructions encoded on a tangible program carrier, for example a computer-readable medium, for execution by, or to control the operation of, a processing system. The computer readable medium can be a machine-readable storage device, a machine-readable storage substrate, a memory device, or a combination of one or more of them.

The term “processing system” encompasses all apparatus, devices, and machines for processing data, including by way of example a programmable processor, a computer, or multiple processors or computers. The processing system can include, in addition to hardware, code that creates an execution environment for the computer program in question, e.g., code that constitutes processor firmware, a protocol stack, a database management system, an operating system, or a combination of one or more of them.

A computer program (also known as a program, software, software application, script, or code) can be written in any form of programming language, including compiled or interpreted languages, or declarative or procedural languages, and it can be deployed in any form, including as a stand-alone program, a module, component, subroutine, or other unit suitable for use in a computing environment. A computer program does not necessarily correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program can be deployed to be executed on one computer or on multiple computers that are located at one site or distributed across multiple sites and interconnected by a communication network.

Computer readable media suitable for storing computer program instructions and data include all forms of non-volatile memory, media and memory devices, including by way of example, semiconductor memory devices, e.g., EPROM, EEPROM, and flash memory devices; magnetic disks, e.g., internal hard disks or removable disks; magneto optical disks; and CD ROM and DVD ROM disks. The processor and the memory can be supplemented by, or incorporated in, special purpose logic circuitry.

Implementations of the subject matter described in this specification can be implemented in a computing system that includes a back end component, e.g., a data server, or that includes a middleware component, e.g., an application server, or that includes a front end component, e.g., a client computer having a graphical user interface or a Web browser through which a user can interact with an implementation of the subject matter described is this specification, or any combination of one or more such back end, middleware, or front end components. The components of the system can be interconnected by any form or medium of digital data communication, e.g., a communication network. Examples of communication networks include a local area network (“LAN”) and a wide area network (“WAN”), e.g., the Internet.

The computing system can include clients and servers. A client and server are generally remote from each other and typically interact through a communication network. The relationship of client and server arises by virtue of computer programs running on the respective computers and having a client server relationship to each other.

While this specification contains many specific implementation details, these should not be construed as limitations on the scope of what may be claimed, but rather as descriptions of features that may be specific to particular implementations. Certain features that are described in this specification in the context of separate implementations can also be implemented in combination in a single implementation. Conversely, various features that are described in the context of a single implementation can also be implemented in multiple implementations separately or in any suitable subcombination. Moreover, although features may be described above as acting in certain combinations and even initially claimed as such, one or more features from a claimed combination can, in some cases, be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the implementations described above should not be understood as requiring such separation in all implementations, and it should be understood that the described program components and systems can generally be integrated together in a single software product or packaged into multiple software products.

A number of implementations of the invention have been described. Nevertheless, it will be understood that various modifications may be made without departing from the spirit and scope of the technology described in this document. Accordingly, other implementations are within the scope of the following claims.

What is claimed is:

1. A weight-bearing device comprising:
a weight-bearing surface configured to bear the weight of a subject;
a first sensor module disposed in the device, the first sensor module configured to measure information about pulse waves propagating through blood in the subject, the subject located in contact with the weight-bearing surface;
a second sensor module disposed in the device, the second sensor module configured to measure information about a motion of the subject; and
a processing device configured to:
receive a first dataset representing time-varying information about at least one pulse wave propagating through blood in the subject, wherein the time-varying information about the at least one pulse wave is measured using the first sensor module;
receive a second dataset representing information about a time-varying motion of the subject, wherein the information about the time-varying motion is measured using the second sensor module;
identify a first point in the first dataset, the first point representing an arrival time of the pulse wave at a first body part of the subject;
identify a second point in the second dataset, the second point representing an earlier time at which the pulse wave traverses a second body part of the subject; and
compute a pulse transit time (PTT) as a difference between the first and second points, the PTT representing a time taken by the pulse wave to travel from the second body part to the first body part of the subject.

2. The device of claim 1, wherein the weight-bearing surface is flexible.

3. The device of claim 2, wherein the second sensor module includes a strain gauge.

4. The device of claim 2, wherein the second sensor module includes a motion sensor.

5. The device of claim 4, wherein the motion sensor includes one or both of an accelerometer and a gyroscope.

6. The device of claim 1, wherein the weight-bearing surface is rigid.

7. The device of claim 1, wherein the second sensor module includes a pressure sensor.

8. The device of claim 1, further comprising a mechanism affixed to an underside of the weight-bearing surface, the mechanism configured to permit the weight-bearing surface to depress.

9. The device of claim 8, wherein the mechanism is a spring.

10. The device of claim 8, wherein the second sensor module includes a motion sensor.

11. The device of claim 10, wherein the motion sensor includes one or both of an accelerometer and a gyroscope.

12. The device of claim 1, wherein the first sensor module includes a light source and an optical sensor.

13. The device of claim 12, wherein the light source is an LED.

14. The device of claim 12, wherein the optical sensor is a photodiode.

15. The device of claim 1, wherein the first sensor module includes an impedance sensor.

16. The device of claim 15, wherein the impedance sensor includes two electrodes positioned less than 4 inches of each other.

17. The device of claim 16, wherein the electrodes are positioned such that a part of the skin of the subject makes direct contact with both of the electrodes when the weight-bearing surface bears the weight of the subject.

18. The device of claim 17, wherein the electrodes are positioned such that a foot of the subject makes direct contact with both of the electrodes when the weight-bearing surface bears the weight of the subject.

19. The device of claim 15, wherein the impedance sensor includes two electrodes positioned greater than or equal to 4 inches from each other.

20. The device of claim 19, wherein the electrodes are positioned such that a first foot of the subject makes contact with one of the electrodes and a second foot of the subject makes contact with the other electrode when the weight-bearing surface bears the weight of the subject.

21. The device of claim 1, wherein the information about pulse waves propagating through blood in the subject comprises photoplethysmographic (PPG) data.

22. The device of claim 1, wherein the information about pulse waves propagating through blood in the subject comprises bio-impedance data.

23. The device of claim 1, wherein the information about a motion of the subject comprises ballistocardiogram (BCG) data.

24. The device of claim 1, wherein the information about a motion of the subject comprises seismocardiogram (SCG) data.

25. The device of claim 1, wherein identifying the first point in the first dataset includes identifying a reference point within the first dataset.

26. The device of claim 25, wherein the reference point is a local maximum, a local minimum, a zero-crossing, or a local maximum of a first derivative within the first dataset.

27. The device of claim 25, wherein the reference point is within an expected range of one or both of time and amplitude.

28. The device of claim 1, wherein identifying the second point in the second dataset includes identifying a reference point within the second dataset.

29. The device of claim 28, wherein the reference point is a local maximum, a local minimum, a zero-crossing, or a local maximum of a first derivative within the first dataset.

30. The device of claim 28, wherein the reference point is within an expected range of one or both of time and amplitude.

31. The device of claim 1, wherein the weight-bearing surface is substantially flat.

32. The device of claim 1, wherein at least one of the first sensor module and the second sensor module is attached to the weight-bearing surface.

33. The device of claim 1, wherein the weight-bearing surface is configured to directly contact the subject when the weight-bearing surface bears the weight of the subject.

34. The device of claim 1, wherein the device is a weight scale.

35. The device of claim 1, wherein the device is integrated into a floor.

36. The device of claim 35, wherein the device is a floor tile.

37. The device of claim 1, wherein the device is a bed.

38. The device of claim 1, wherein the device is a yoga mat.

39. The device of claim 1, wherein the device is a shoe.

40. The device of claim 39, wherein the weight-bearing surface is a sole of the shoe.

41. The device of claim 40, wherein at least one of the first sensor module and the second sensor module is attached to the sole of the shoe.

42. The device of claim 1, wherein the device is a chair.

43. The device of claim 1, wherein the second sensor module includes a sensor for measuring a weight of the subject.

44. The device of claim 1, wherein the processing device is further configured to determine one or more of a blood pressure, a heart rate, a respiratory rate, a blood oxygen level, a stroke volume, a cardiac output, and a temperature of the subject.

45. The device of claim 44, wherein the processing device determines the heart rate, the respiratory rate, the stroke volume, and the cardiac output based on the information measured by the second sensor module without using the information measured by the first sensor module.

46. The device of claim 1, wherein the second sensor module is configured to measure a weight of the subject.

47. The device of claim 15, wherein the device is configured to measure a body composition of the subject.

48. The device of claim 47, wherein the body composition of the subject includes a fat content of the subject.

49. A device comprising:
a weight-bearing surface configured to bear the weight of a subject;
a first sensor module and a second sensor module each disposed in the device, the first sensor module and the second sensor module each configured to measure information about pulse waves propagating through blood in the subject, the subject located in contact with the weight-bearing surface; and
a processing device configured to:
receive a first dataset representing time-varying information about at least one pulse wave propagating through blood in the subject, wherein the time-varying information about the at least one pulse wave is measured using the first sensor module;
receive a second dataset representing time-varying information about the at least one pulse wave propagating through blood in the subject, wherein the time-varying information about the at least one pulse wave is measured using the second sensor module;
identify a first point in the first dataset, the first point representing an arrival time of the pulse wave at a first body part of the subject;
identify a second point in the second dataset, the second point representing an arrival time of the pulse wave at a second body part of the subject; and
compute a pulse transit time (PTT) as a difference between the first and second points, the PTT representing a time taken by the pulse wave to travel from the first body part to the second body part of the subject.

50. The device of claim 49, wherein at least one of the first sensor module and the second sensor module includes a light source and an optical sensor.

51. The device of claim 50, wherein the light source is an LED.

52. The device of claim 50, wherein the optical sensor is a photodiode.

53. The device of claim 49, wherein at least one of the first sensor module and the second sensor module includes an impedance sensor.

54. The device of claim 53, wherein the impedance sensor includes two electrodes positioned less than 4 inches of each other.

55. A device comprising:
a weight-bearing surface configured to bear the weight of a subject;
a first sensor module disposed in the device, the first sensor module configured to measure information about pulse waves propagating through blood in the subject, the subject located in contact with the weight-bearing surface;
a second sensor module disposed in the device, the second sensor module configured to measure information about electrical signals related to the heart of the subject; and
a processing device configured to:
receive a first dataset representing time-varying information about at least one pulse wave propagating through blood in the subject, wherein the time-varying information about the at least one pulse wave is measured using the first sensor module;
receive a second dataset representing time-varying information about electrical signals related to the heart of the subject, wherein the time-varying information about electrical signals related to the heart of the subject is measured using the second sensor module;
identify a first point in the first dataset, the first point representing an arrival time of the pulse wave at a body part of the subject;
identify a second point in the second dataset, the second point representing an earlier time at which the heart of the subject is depolarized, wherein the pulse wave is originated from the heart of the subject in response to the depolarization; and
compute a pulse arrival time (PAT) as a difference between the first and second points, the PAT representing an elapsed time between the pulse wave being originated and the pulse wave arriving at the body part of the subject.

56. The device of claim 55, wherein the PAT represents an approximate time taken by the pulse wave to travel from the heart of the subject to the body part of the subject.
